(19) United States
(12) Patent Application Publication
(10) Pub. No.: US 2025/0380882 A1
(43) Pub. Date: Dec. 18, 2025

(54) METHODS AND DEVICES WITH DETECTION OF MUSCLE FATIGUE ONSET FOR PHYSICAL ACTIVITY MONITORING AND INJURY PREVENTION

(71) Applicant: George Mason University, Fairfax, VA (US)

(72) Inventors: Marissa Ashton Howard, Richmond, VA (US); Lance Allen Liotta, Bethesda, MD (US); Amad Aamir Qureshi, Falls Church, VA (US); Amanda Nicole Haymond Still, Manassas, VA (US)

(21) Appl. No.: 19/236,020

(22) Filed: Jun. 12, 2025

Related U.S. Application Data
(60) Provisional application No. 63/659,462, filed on Jun. 13, 2024.

(51) Int. Cl. A61B 5/11; A61B 5/00

(52) U.S. Cl. CPC A61B 5/1126; A61B 5/746; A61B 2562/0219

(57) ABSTRACT

Methods and systems for providing monitoring and feedback of muscle use involve monitoring frequencies in measurements of acceleration and/or rotation of at least one muscle, detecting high frequency oscillations (HFOs) indicative of muscle fatigue preceding muscle failure in the monitored frequencies, and, in response to the detection of the HFOs, sending an alert before occurrence of muscle failure.

### CROSS-REFERENCE TO RELATED APPLICATIONS

This application claims the benefit of U.S. Provisional Patent App. No. 63/659,462, filed Jun. 13, 2024, the complete contents of which are herein incorporated by reference.

### FIELD OF THE INVENTION

Embodiments generally relate to methods and systems for physical activity monitoring and injury prevention and, more specifically, for detection of and response to muscle fatigue onset.

### BACKGROUND

Muscle fatigue has been implicated in a variety of illnesses such as cardiovascular disease, obesity, cancer, arthritis, osteoarthritis, and post-injury rehabilitation. In particular, neurological illnesses, including Parkinson's disease, multiple sclerosis, myasthenia gravis, traumatic brain injury, and stroke, have a higher prevalence of muscular fatigue symptoms. Additionally, understanding muscle fatigue is of high importance for athletic or military individuals who rely on improving muscular strength and performance.

Muscular fatigue occurs when there is a decrease in the power/force generating capacity of prolonged or repeated muscle activity. This is caused by many underlying biological reasons including but not limited to: loss of oxygen, blood flow changes, neural activity levels, and decreased metabolic substrates and adenosine triphosphate (ATP). Furthermore, performing dynamic exercises that contribute to fatigue stimulates the nervous system to compensate to maintain performance. However, over time, central fatigue occurs and the feedback to compensate the onset of fatigue weakens.

Present methods to detect muscular fatigue rely on measuring the electrical outputs of electrically stimulated “fatigued” versus “non-fatigued” muscles. These methods require advanced equipment and fail to provide a real-time, longitudinal measurement of a muscle becoming fatigued following activity.

Currently there is no simple and effective method for the collection and analysis of biomechanical data of joints and muscles that can determine patient progress for rehabilitation and act as an aid for early diagnosis of complications within the neuromuscular skeletal system.

### SUMMARY

Embodiments of this disclosure detect the onset of fatigue by identifying neural-muscular feedback failure before total fatigue occurs. A high-resolution inertial monitoring unit (IMU) may be used to analyze dynamic contractions of a movement such as but not limited to a cyclic or repetitive exercise in at least one degree of freedom. High frequency oscillations (HFOs) are detected at the onset of muscle fatigue or loss of muscle power.

An embodiment embeds IMU sensor(s) in exercise equipment, such as a barbell, to detect the HFOs prior to fatigue or muscle failure after cyclic or repetitive movement. According to an embodiment, a user may be notified before an exercise injury. Some embodiments may track the performance of muscle(s) over time. Overall, the technology may identify HFOs of a fatiguing muscle. The HFOs may be utilized to rehabilitate and support various individuals suffering from muscular fatigue.

According to an embodiment, IMU sensor(s) may be integrated (or used) with various products that contact the skin. According to an embodiment, IMU sensor(s) may be integrated (or used) with various products that allow body measurements such as described herein, to be detected. According to an embodiment, IMU sensor(s) may be integrated (or used) with various devices. For example, IMU sensor(s) may be integrated with exercise equipment. According to an embodiment, IMU sensor(s) may be integrated with handheld tools. According to an embodiment, IMU sensor(s) may be integrated with clothing such as exercise pants, shirts, socks, gloves, and/or shoes. According to an embodiment, IMU sensor(s) may be integrated with wearable devices such as a watch, bracelet, anklet, arm band, and/or leg band.

According to an embodiment, a sensor utilized to detect muscle fatigue may comprise an inertial monitor unit. The IMU may measure movement in at least 3 degrees of freedom. The sampling rate of the sensor may be at least 9600 Hz to achieve the sensitivity of fatigue detection. During dynamic muscle contractions, the IMU may return a cyclic continuous wave pattern.

The onset of fatigue may be determined in numerous ways by analyzing wave pattern changes. For example, according to an embodiment, the onset of fatigue may be determined when the waveform changes such that the period (T) between peaks in the X and Y planes increases due to increased time to complete a dynamic contraction. When the period T increases from the previous average of periods (T_{A}) by greater then but not limited to 5%, muscular fatigue may have begun. According to an embodiment, the onset of fatigue may be determined when the waveform changes such that given an oscillation that has a peak frequency of F, if the peak frequency oscillation is greater than but not limited to 5% of the rolling average of the previous 2 peak frequency oscillations. According to an embodiment, the onset of fatigue may be determined when the waveform changes such that the fundamental frequency of a given oscillation is greater than but not limited to a rate of 5% compared to the rolling average fundamental frequencies of the previous 10 oscillations. According to an embodiment, the onset of fatigue may be determined when the waveform changes such that a given signal has a power frequency with a peak that is at least, but not limited to, 2-fold over baseline. Additionally, a lack of baseline may be also indicative of complete muscle failure.

For example, according to an embodiment, the onset of fatigue may be determined when the waveform changes such that the period (T) between peaks in the X and Y planes increases due to increased time to during static contractions. When the period T increases from the previous average of periods (T_{A}) by greater then but not limited to 5%, muscular fatigue may have begun.

According to an embodiment, a sensor may be embedded or enclosed in a ball. According to an embodiment, a sensor may be embedded or enclosed in exercise equipment. According to an embodiment, a sensor may be embedded or enclosed in exercise equipment or tool. According to an embodiment, a sensor may attach to a wearable device. According to an embodiment, the sensor may non-invasively attach to the wearable device. According to other embodiments, the sensor may be attached to other items that may be used in recreational equipment or rehabilitation equipment or exercise equipment or training equipment or therapy devices.

According to an embodiment, a system may monitor rehabilitation and/or progress of therapy over time to detect muscle strength and/or performance changes over time. According to an embodiment, the system self-calibrates the total range of motion and speed of motion during baseline measurements.

According to an embodiment, the system may detect muscle fatigue onset. According to an embodiment, muscle fatigue onset may be determined employing data analysis. According to an embodiment, data may comprise the measurement(s). According to an embodiment, data may comprise motion measurement(s). According to an embodiment, data may comprise muscle feedback measurement(s). According to an embodiment, data may comprise accelerometer measurement(s). According to an embodiment, data may comprise IMU measurement(s).

According to an embodiment, the analysis may comprise determining frequency characteristics of the data. According to an embodiment, the analysis may comprise determining time domain characteristics of the data. According to an embodiment, the analysis may comprise determining time variant characteristics of the data.

According to an embodiment, the system generates a notification when muscle fatigue onset may have been detected. According to an embodiment, the system generates a notification when muscle fatigue onset has been detected. According to an embodiment, the device may send a notification employing wireless connectivity. According to an embodiment, the wireless connectivity may comprise Wi-Fi. According to an embodiment, the wireless connectivity may comprise Bluetooth. According to an embodiment, the wireless connectivity may comprise cellular communications. According to an embodiment, the wireless connectivity may comprise optical connectivity.

According to an embodiment, the device may send data for storage into the cloud. According to an embodiment, the data may be processed in the cloud. According to an embodiment, the data may be processed externally. According to an embodiment, the processing may comprise muscle fatigue onset. According to an embodiment, data may be assembled by a mobile application. According to an embodiment, the data may be visualized. According to an embodiment, the visualization may comprise analysis features. According to an embodiment, the analysis features may be configured for easy readability.

According to an embodiment, an exemplary user may be a mammal. The mammal may be a human or may be a simian, or may be a canine, or may be a feline or may be an equine.

According to an embodiment, the onset of muscle fatigue may be detected in a mammal. According to an embodiment, the mammal may be a human. According to an embodiment, the mammal may be a simian. According to an embodiment, the mammal may be a canine.

According to an embodiment, the mammal may be a feline. According to an embodiment, the mammal may be an equine.

According to an embodiment, measurements may be obtained from a sensor. According to an embodiment, the sensor is configured to measure at least one muscle contraction of a mammal. According to an embodiment, the measurements may be represented as data.

According to an embodiment, the measurements may be about one axis of rotation. According to an embodiment, the measurements are about one axis of rotation. According to an embodiment, the measurements may be in at least one axis of movement of the mammal. According to an embodiment, the measurements may be about two axes of rotation. According to an embodiment, the measurements are about two axes of rotation. According to an embodiment, the measurements are about two axes of rotation of movement of a limb of a mammal. According to an embodiment, the measurements may be about three axes of rotation. According to an embodiment, the measurements are about three axes of rotation. According to an embodiment, the measurements are about three axes of rotation of movement of a limb of a mammal.

According to an embodiment, the onset of fatigue may be determined in numerous ways by analyzing wave pattern changes. According to an embodiment, the onset of fatigue may be determined in numerous ways by analyzing wave pattern changes to maintain a static position for a mammal. According to an embodiment, the onset of fatigue may be determined in numerous ways by analyzing wave pattern changes to maintain a static position of a limb a mammal. According to an embodiment, the onset of fatigue may be determined in numerous ways by analyzing wave pattern changes to maintain a static position of multiple limbs of a mammal.

According to an embodiment, the measurement may be a period (T) of movement data corresponding to muscle activity required to maintain a static position for a mammal. According to an embodiment, the measurement may be a period (T) of movement data corresponding to muscle activity required to maintain a static position of a limb of a mammal. According to an embodiment, the measurement may be a period (T) of movement data corresponding to muscle activity required to maintain a static position of multiple limbs of a mammal. According to an embodiment, the measurement may be a period (T) of movement data corresponding to muscle activity required to complete the movement of a limb of a mammal.

According to an embodiment, muscle fatigue may be identifying when the period T increases from the previous average of periods (T_{A}). According to an embodiment, the period T increase from the previous average of periods (T_{A}) may be greater than a determined percentage. According to an embodiment, the onset of muscle fatigue is determined when the period T increases from the previous average of periods (T_{A}) by greater then but not limited to five percent. According to an embodiment, the determined percentage may be predetermined. According to an embodiment, the determined percentage may be determined dynamically. According to an embodiment, the determined percentage may be greater than five percent. According to an embodiment, the determined percentage may be greater than ten percent. According to an embodiment, the determined percentage may be greater than fifteen percent. According to an embodiment, the onset of muscle fatigue is determined when the period T increases from the previous average of periods (T_{A}) by greater then but not limited to five percent for a dynamic contraction. According to an embodiment, the onset of muscle fatigue is determined when the period T increases from the previous average of periods (T_{A}) by greater then but not limited to five percent for a dynamic contraction to maintain a static position. According to an embodiment, the onset of muscle fatigue is determined when the period T increases from the previous average of periods (T_{A}) by greater then but not limited to five percent for a dynamic contraction to maintain a static position of a limb of a mammal. According to an embodiment, the onset of muscle fatigue is determined when the period T increases from the previous average of periods (T_{A}) by greater then but not limited to five percent for a dynamic contraction to maintain a static position of multiple limbs of a mammal. According to an embodiment, the onset of muscle fatigue is determined when the period T increases from the previous average of periods (T_{A}) by greater then but not limited to five percent for a dynamic contraction for the movement of a limb of a mammal.

According to an embodiment, an average of periods (T_{A}) may comprise an average of at least 3 periods. According to an embodiment, an average of periods (T_{A}) may comprise an average of at least 3 periods to maintain a static position for a mammal. According to an embodiment, an average of periods (T_{A}) may comprise an average of at least 3 periods of activity to maintain a static position of a limb of a mammal. According to an embodiment, an average of periods (T_{A}) may comprise an average of at least 3 periods of activity to maintain a static position of multiple limbs of a mammal. According to an embodiment, an average of periods (T_{A}) may comprise an average of at least 3 periods of activity for a dynamic contraction for the dynamic contraction for the movement of a limb of a mammal.

According to an embodiment, the onset of muscle fatigue is determined when the period (T) between peaks in the X and Y planes increases due to increased time to complete a dynamic contraction. According to an embodiment, the onset of muscle fatigue is determined when the period (T) between peaks in the X and Y planes increases due to increased time of a dynamic contraction to maintain a static position. According to an embodiment, the onset of muscle fatigue is determined when the period (T) between peaks in the X and Y planes increases due to increased time of a dynamic contraction to maintain a static position of a limb of a mammal. According to an embodiment, the onset of muscle fatigue is determined when the period (T) between peaks in the X and Y planes increases due to increased time of a dynamic contraction to maintain a static position of multiple limbs of a mammal. According to an embodiment, the onset of muscle fatigue is determined when the period (T) between peaks in the X and Y planes increases due to increased time of a dynamic contraction for the movement of a limb of a mammal.

According to an embodiment, the onset of muscle fatigue is determined when given an oscillation that has a peak frequency of F, if the peak frequency oscillation is greater than but not limited to five percent of the rolling average of the previous two peak frequency oscillations. According to an embodiment, the onset of muscle fatigue is determined when given an oscillation that has a peak frequency of F, if the peak frequency oscillation is greater than but not limited to five percent of the rolling average of the previous two peak frequency oscillations to maintain a static position. According to an embodiment, the onset of muscle fatigue is determined when given an oscillation that has a peak frequency of F, if the peak frequency oscillation is greater than but not limited to five percent of the rolling average of the previous two peak frequency oscillations to maintain a static position of a limb of a mammal. According to an embodiment, the onset of muscle fatigue is determined when given an oscillation that has a peak frequency of F, if the peak frequency oscillation is greater than but not limited to five percent of the rolling average of the previous two peak frequency oscillations to maintain a static position of multiple limbs of a mammal. According to an embodiment, the onset of muscle fatigue is determined when given an oscillation that has a peak frequency of F, if the peak frequency oscillation is greater than but not limited to five percent of the rolling average of the previous two peak frequency oscillations for a dynamic contraction for the movement of a limb of a mammal.

According to an embodiment, the sensor may comprise an accelerometer. According to an embodiment, the sensor is an accelerometer. An accelerometer is a device that measures the acceleration of an object.

A basic mechanical accelerometer may comprise a damped proof mass on a spring. When the accelerometer experiences an acceleration, Newton's third law causes the spring's compression to adjust to exert an equivalent force on the mass to counteract the acceleration. Since the spring's force scales linearly with amount of compression (according to Hooke's law) and because the spring constant and mass are known constants, a measurement of the spring's compression is also a measurement of acceleration. The system may be damped to prevent oscillations of the mass and spring interfering with measurements. However, the damping causes accelerometers to have a frequency response.

Many animals have sensory organs to detect acceleration, especially gravity. In these, the proof mass is usually one or more crystals of calcium carbonate otoliths (Latin for “ear stone”) or statoconia, acting against a bed of hairs connected to neurons. The hairs form the springs, with the neurons as sensors. The damping is usually by a fluid. Many vertebrates, including humans, have these structures in their inner ears. Most invertebrates have similar organs, but not as part of their hearing organs. These are called statocysts.

Mechanical accelerometers are often designed so that an electronic circuit senses a small amount of motion, then pushes on the proof mass with some type of linear motor to keep the proof mass from moving far. The motor might be an electromagnet or in very small accelerometers, electrostatic. Since the circuit's electronic behavior can be carefully designed, and the proof mass does not move far, these designs may be stable (i.e. they do not oscillate), very linear with a controlled frequency response. This may be called servo mode design.

In mechanical accelerometers, measurement is often electrical, piezoelectric, piezoresistive or capacitive. Piezoelectric accelerometers use piezoceramic sensors (e.g. lead zirconate titanate) or single crystals (e.g. quartz, tourmaline). They are unmatched in high frequency measurements, low packaged weight, and resistance to high temperatures. Piezoresistive accelerometers resist shock (very high accelerations) better. Capacitive accelerometers typically use a silicon micro-machined sensing element. They measure low frequencies well.

Modern mechanical accelerometers may comprise small micro-electro-mechanical systems (MEMS), and are often very simple MEMS devices, consisting of little more than a cantilever beam with a proof mass (also known as seismic mass). Damping results from the residual gas sealed in the device. As long as the Q-factor is not too low, damping may not result in a lower sensitivity.

Under the influence of external accelerations, the proof mass may deflect from its neutral position. This deflection may be measured in an analog or digital manner. Most commonly, the capacitance between a set of fixed beams and a set of beams attached to the proof mass is measured. This method is simple, reliable, and inexpensive. Integrating piezoresistors in the springs to detect spring deformation, and thus deflection, may be an alternative, although a few more process steps may be needed during the fabrication sequence. For very high sensitivities quantum tunnelling may also be used. Optical measurements may also be used to measure acceleration.

Another MEMS-based accelerometer is a thermal (or convective) accelerometer. It may contain a small heater in a very small dome. This heats the air or other fluid inside the dome. The thermal bubble acts as the proof mass. An accompanying temperature sensor (like a thermistor; or thermopile) in the dome measures the temperature in one location of the dome. This may measure the location of the heated bubble within the dome. When the dome is accelerated, the colder, higher density fluid may push the heated bubble. The measured temperature changes. The temperature measurement is interpreted as acceleration. The fluid provides the damping. Gravity acting on the fluid provides the spring. Since the proof mass may be a very lightweight gas, and not held by a beam or lever, thermal accelerometers may survive high shocks. Another variation may use a wire to both heat the gas and detect the change in temperature. The change of temperature changes the resistance of the wire. A two dimensional accelerometer can be economically constructed with one dome, one bubble and two measurement devices.

Some micromechanical accelerometers operate in-plane, that is, they are designed to be sensitive only to a direction in the plane of a surface. By integrating two devices perpendicularly on a single surface a two-axis accelerometer can be made. By adding another out-of-plane device, three axes can be measured. Such a combination may have lower misalignment error than three discrete models combined after packaging. Micromechanical accelerometers may be available in a wide variety of measuring ranges.

According to an embodiment, the accelerometer may be configured to take measurements of muscle contractions. According to an embodiment, the accelerometer may be configured to take measurements of muscle movement. According to an embodiment, the accelerometer configuration may comprises embedding the accelerometer in a device that is physically moved by muscle activity. According to an embodiment, the accelerometer configuration may comprises embedding the accelerometer in clothing that is physically moved by muscle activity. According to an embodiment, the accelerometer configuration may comprises embedding the accelerometer in wearable device that is physically moved by muscle activity. According to an embodiment, the accelerometer may be configured to determine of the periods of the accelerometer measurements. According to an embodiment, the accelerometer may be configured to determine of the average of periods of the accelerometer measurements.

According to an embodiment, the determination of the period and average of periods may be conducted on a device that may be linked to the sensor. According to an embodiment, the determination of the period and average of periods is conducted on a device that is linked to the sensor. According to an embodiment, the device may be a smart watch. According to an embodiment, the device may is a smart watch. According to an embodiment, the device may be a cell phone. According to an embodiment, the device may be a cloud computing system. According to an embodiment, the sensor may be an accelerometer. According to an embodiment, the accelerometer may be part of a smart watch. According to an embodiment, the accelerometer may be part a cell phone.

According to an embodiment, the sensor may be embedded in a weighted object. According to an embodiment, the weighted object may be a barbell. According to an embodiment, the weighted object may be a dumbbell. According to an embodiment, the weighted object may be a weighted exercise ball. According to an embodiment, the weighted object may be a strength training machine. According to an embodiment, the weighted object may be at least one end of a weighted rope. According to an embodiment, the weighted object may be a mammal wearable weighted object. According to an embodiment, the wearable weighted object may be an ankle weight. According to an embodiment, the wearable weighted object may be a leg weight. According to an embodiment, the wearable weighted object may be a wrist weight. According to an embodiment, the weighted object may be a tool.

According to an embodiment, the tool may be a power tool. According to an embodiment, the tool may be a battery powered tool. According to an embodiment, the tool may be a handheld tool. According to an embodiment, the tool may be a hydraulic tool. According to an embodiment, the tool is a pneumatic tool. According to an embodiment, the weighted object may be a durable medical device. According to an embodiment, the weighted object may be a wearable durable medical device.

According to an embodiment, the accelerometer sampling rate may be at least 9600 Hz. According to an embodiment, the accelerometer sampling rate is at least 9600 Hz.

According to an embodiment, the dynamic contraction may be an isolated muscle. According to an embodiment, the dynamic contraction is an isolated muscle. According to an embodiment, the dynamic contraction may be a group of muscles. According to an embodiment, the dynamic contraction is a group of muscles.

According to an embodiment, a noninvasive system includes an embedded 6 axis IMU (accelerometer and gyroscope) to measure muscular activity over time and discover previously unknown distinguishing characteristics of muscular fatigue.

According to an embodiment, muscular fatigue can be characterized in the signal (unfiltered and/or filtered) as a combination with changes in amplitude of the oscillations within a period, frequency of oscillation within the period, and time delay between periods. Combining the data analysis with the overall system and minimizing delay in data transfer and storage, the system is an effective medium for early diagnosis and for rehabilitative care among patients with neuromuscular skeletal complications.

According to some exemplary embodiments, frequencies monitored and assessed for recognizing the onset of muscle fatigue are frequencies generated by neural signals but which are detectable using accelerometer(s) and/or gyroscope(s). The frequencies are caused by feedback control of two or more opposing muscle groups causing a movement or lack thereof in three-dimensional (3D) space of one or more muscle groups. These frequencies are generated by neural signals trying to control the muscle group movement (e.g., via feedback loop).

According to some embodiments, whether muscle fatigue exists may be determined only from one or more acceleration signals, only from one or more rotation signals, or only from a combination of acceleration and rotation signals. Despite some frequencies of interest having origins in neural signals, some exemplary embodiments do not use any electrical activity signals such as but not limited to electromyogram (EMG) signals to determine a state of fatigue of a muscle or muscle group.

According to some exemplary embodiments, high frequency oscillations include frequencies above 80 Hz. According to some exemplary embodiments, high frequency oscillations are frequencies above 80 Hz. According to some exemplary embodiments, high frequency oscillations include frequencies of 100 Hz and greater. According to some exemplary embodiments, high frequency oscillations are frequencies of 100 Hz and greater. According to some embodiments, one or more thresholds distinguishing HFOs from oscillations which do not qualify as HFOs may be some number other than 80 or 100 Hz. The shape of some waveforms of interest for some embodiments shows that these are characteristic of moving muscles under an applied load. Different muscle groups can have different characteristics (e.g. type of muscle, length of the muscle, where muscles are attached to bone, speed of the motion). One or more of these considerations may be used in some embodiments to determine a threshold only frequencies above which qualify as HFOs. In some embodiments, one or more qualities of the subject being monitored for HFOs may be used in the determination of a threshold only frequencies above which qualified as HFOs. The one or more qualities may include the species of the subject. For example, some embodiments may determine a different threshold for a human subject than for a non-human subject (e.g., an equestrian subject). For equestrian applications, the numbers may be different than for human applications.

Exemplary embodiments include a minimalistic sleeve for monitoring biomechanics. The sleeve may be universal. The sleeve may be configured to monitor biomechanics of joints including but not limited to arms knee and ankle. A universal sleeve to monitor biomechanical activity may include a sleeve (including microcontroller), companion app, cloud computing infrastructure, and an analysis toolkit.

According to an embodiment, a device may include an accelerometer and gyroscope. Further components of exemplary systems may include but are not limited to an app, cloud, microcontroller, and phone. Some embodiments may include the configuration stage which may involve the use of training datasets to improve automated signal processing and feature extraction.

Exemplary embodiments include a method for determining deterioration or improvement in muscular strength/activity. Signal analysis including but not limited to feature extraction may include consideration of features such as amplitude, frequency, and time delay between periods. Components of exemplary systems may be integral with one another e.g. a sleeve with the microcontroller and receiver/transmitter hardware. In addition or in the alternative, components of exemplary systems may be separate and distinct from one another with means for communication and communication protocols between the components. One example approach for the data transfer: data is sent from wearable device via BLE to phone, and data is pushed from the phone to a cloud server.

Some embodiments include a novel minimalistic biomechanic monitoring speed system for use by but not limited to patients with joint and muscular injuries or at risk of joint or muscular injuries. Embodiments include a universal system to monitor muscular activity of users such as but not limited to patients undergoing (neuro) muscular rehabilitation.

According to some embodiments, determination/recognition of muscle fatigue and muscle fatigue onset is not determined from any bulk movement data.

According to some embodiments, determination/recognition of muscle fatigue and muscle fatigue onset is not determined from any camera, ultrasound, or electromyogram (EMG) recordings/data.

### BRIEF DESCRIPTION OF THE DRAWINGS

FIG. 1 is sample acceleration data collected from a muscle in use as the muscle progresses from a no fatigue state to fatigue onset and then to muscle failure.

FIG. 2 is sample acceleration data collected from a lower arm of a human subject as the lower arm performed bicep curls.

FIG. 3 is rotation data collected from a lower arm of a human subject as the lower arm performed bicep curls.

FIG. 4 is further sample acceleration data collected from a lower arm of a human subject as the lower arm performed bicep curls.

FIG. 5 is further sample rotation data collected from a lower arm of a human subject as the lower arm performed bicep curls.

FIG. 6 is a schematic of an exemplary system.

FIG. 7 is a depiction of a human user with an exemplary wearable device on the arm monitoring acceleration and/or rotation of the arm over time.

FIG. 8 is a depiction of a human arm with alternative placement positions for an exemplary wearable device.

FIG. 9 is a flowchart of exemplary data processing and resultant determinations.

FIG. 10 is an illustration of assessment of periods of a sample acceleration data stream.

FIG. 11 is an exemplary display device with a graphical user interface (GUI) for providing alerts and access to a user.

FIGS. 12A, 12B, and 12C are diagrams of alternative exemplary system configurations.

FIGS. 13A, 13B, and 13C are diagrams of further alternative exemplary system configurations.

### DETAILED DESCRIPTION

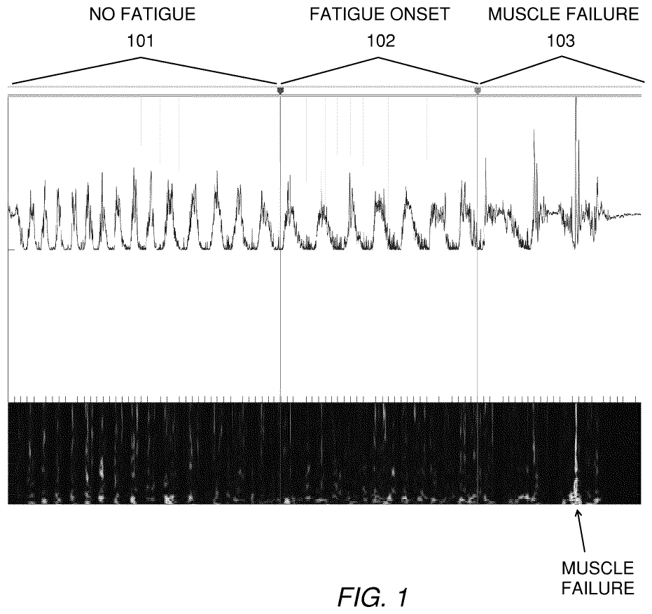

FIG. 1 depicts movement data (in this case acceleration data) collected from a muscle of a subject as the muscle is being used. A significant feature underlying exemplary embodiments of this disclosure is the use of movement data like that which is depicted in FIG. 1 to detect (recognize) the onset of muscle fatigue and anticipate muscle failure before occurrence of muscle failure. The movement data in FIG. 1 characterizes movement which in general is not necessarily detectable with the human eye. A discovery underlying present embodiments is that muscles have very small translational and rotational displacements which in some embodiments may be characterized as imperceptible vibration or tremor. Such displacements are cyclic/oscillating in nature. Exemplary embodiments monitor frequencies in acceleration or rotation measurements of at least one muscle. Changes in the frequency behavior of a muscle, muscle group, or limb is used by exemplary embodiments to identify occurrence of muscle fatigue prior to muscle failure.

In FIG. 1 portion 101 is a sample of recorded movement data which an exemplary device recognizes as indicative of no (muscle) fatigue. Portion 102 is a sample of recorded movement data which an exemplary device recognizes as indicative of fatigue onset. Portion 103 is a sample of recorded movement data which an exemplary device recognizes as indicative of muscle failure. Data segments 101 and 102 both show cyclic sinusoidal patterns. However, the period T of the oscillation increases when fatigue begins to onset. This and other high frequency oscillation features may be used in exemplary embodiments to recognize the onset of muscle fatigue prior to muscle failure. The bottom of FIG. 1 is a scalogram which shows a dramatic increase ripple of the frequency at muscle failure. Exemplary embodiments bear the advantage of providing warning of imminent muscle failure at an existing activity level prior to actual muscle failure.

A significant benefit of some exemplary embodiments of this disclosure is the means to alert a user of the onset of muscle fatigue before occurrence of muscle failure. So alerted, a user may desist from further strenuous use of the muscle which if continued would have led to imminent muscle failure. In general, muscle failure is to be avoided. Muscle failure may entail physical tearing of muscle tissue which is a direct physical injury. Muscle failure can also lead to indirect physical injuries. For example, a subject lifting a heavy object the time of muscle failure may involuntarily lose control of the heavy object. The heavy object may fall an impact or crush some part of the subject or a bystander. Loss of control of a heavy object can also cause physical damage to surroundings. Avoidance or prevention of muscle failure is therefore highly desirable.

Movement data which may be measured and monitored and subject to feature extraction in exemplary embodiments of this disclosure include linear movement data and rotational movement data. Linear movement data includes linear displacement and linear acceleration. Rotational movement data includes rotational displacement and rotational acceleration. Linear movement data may be collected using one or more sensors such as accelerometers. Rotational movement data may be collected using one or more sensors such as gyroscopes.

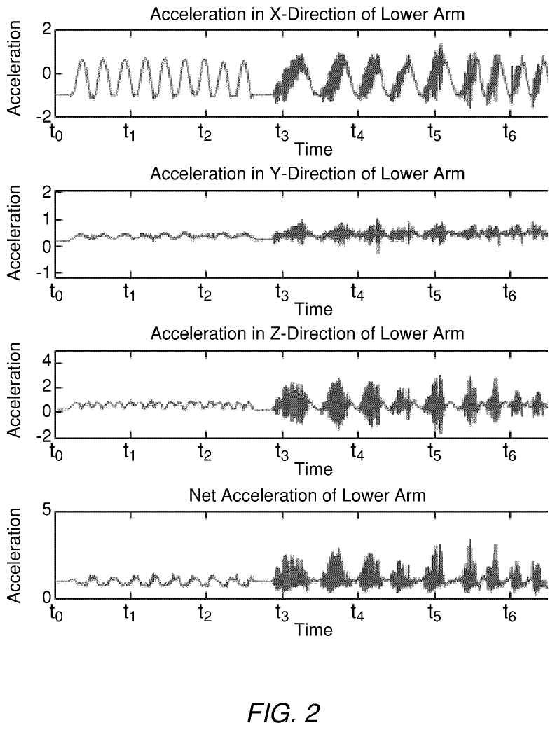

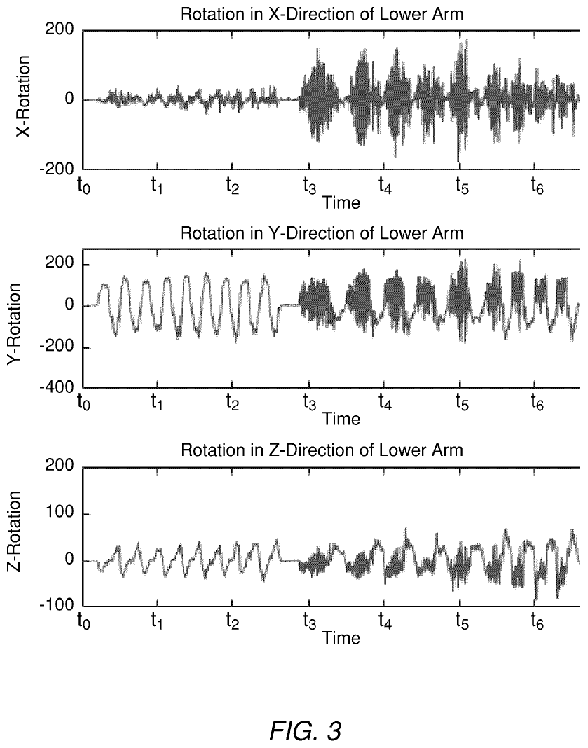

FIGS. 2 and 3 are sample acceleration data and rotation data, respectively, collected from a lower arm of a human subject as the lower arm performed curls. The extracted signals generally depict sinusoidal curves with some noise but with relatively smooth characteristics from time t₀ to just before t₃. These signals are interpreted by exemplary devices and methods as indicative of regular activity by unfatigued muscles. By contrast, the movement data for both acceleration and rotation notably shifts in waveform characteristics beginning just before time t₃. From this point forward exemplary devices and methods would recognize the samples signals as indicative of muscle fatigue. The extracted signals still show generally sinusoidal waveforms (indicative that muscle failure has not yet been sustained) but with many high frequency oscillations superimposed on the general sinusoid. In addition, many of the signal feeds show changes in period and amplitude. The individual performing the curls was fatigued, tired, and/or overstressing his or her muscles.

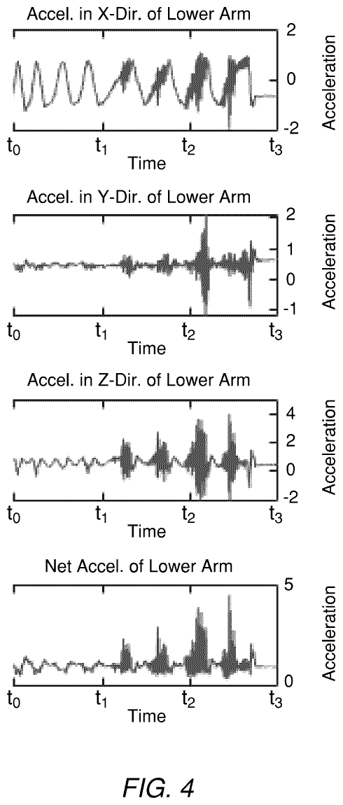

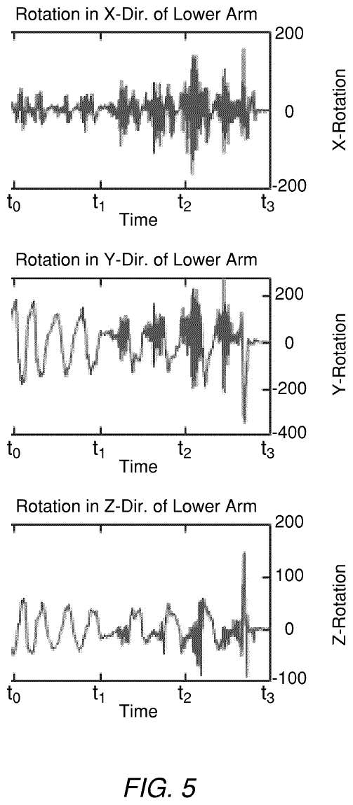

FIGS. 4 and 5 are further samples of acceleration data and rotation data, respectively, collected from a lower arm of a human subject as the lower arm performed curls. These figures illustrate that biomechanic signals expressly monitored and analyzed according to exemplary embodiments of this disclosure may not only recognize the onset of muscle fatigue but also differentiate between multiple differing levels of muscle fatigue. Exemplary embodiments processing the data of FIGS. 4 and 5 may be configured to recognize normal muscle fatigue from approximately time t₀ to t₁. A first stage of muscle fatigue is determined from the signals for time interval approximately t₁ to t₂. A second more extreme stage of muscle fatigue is determined from the signals from approximate time t₂ and up.

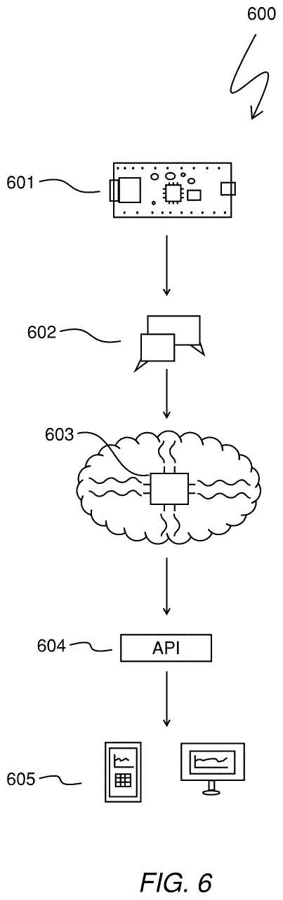

FIG. 6 is a schematic of an exemplary system 600. The system 600 comprises one or more sensors 601 and one or more processors 603. The one or more sensors 601 are configured to measure acceleration and/or rotation of at least one muscle. The one or more processors 603 are configured to monitor frequencies in the measurements of acceleration and/or rotation of the at least one muscle; detect high frequency oscillations (HFOs) indicative of muscle fatigue preceding muscle failure in the monitored frequencies; and in response to the detection of the HFOs, send an alert before occurrence of muscle failure. Processors 603 may be locally situated with sensors 601, remote (e.g., processors accessed through cloud networking), or some combination of these. Cloud computing may be used for functionalities such as but not limited to data storage and data analysis. The system 600 may further include one or more feedback devices 605 configured to produce one or more of a haptic, audial, and visual signal for conveying the alert to a user.

The one or more sensors 601 may be configured together in at least one inertial measurement unit (IMU). In general, sensors 601 include at least one accelerometer configured to measure acceleration and/or at least one gyroscope configured to measure rotation. The one or more sensors 601 may be configured together as an IMU and/or be integrally produced with one or more microcontrollers. A microcontroller may include sensor integration (e.g., integral IMU). A microcontroller may include communication protocols such as but not limited to Wi-Fi and/or Bluetooth low energy (BLE).

One or more applications (i.e., apps) may run on a device 605. An exemplary app may be configured with functionalities such as but not limited to receiving data, displaying data of sensors, local storage, and/or sending data to cloud. An app may be used to bridge a communications gap between microcontroller(s) and cloud server(s). The app and its associated hardware running the app may be used to, for example, retrieve data from the cloud and retrieve data from BLE to send to the cloud.

Various communication means and protocols 602 and 604 may be used for the transfer of information between components of system 600. As non-limiting examples, the sensors 601 may have their data transferred to one or more processors 603 using a messaging protocol 602 such as MQTT (Message Queuing Telemetry Transport). As a further non-limiting example, data from one or more processors 602 may be handled prior to display or other output from device(s) 605 using an application programming interface (API) such as but not limited to REST API (Representational State Transfer API) 604.

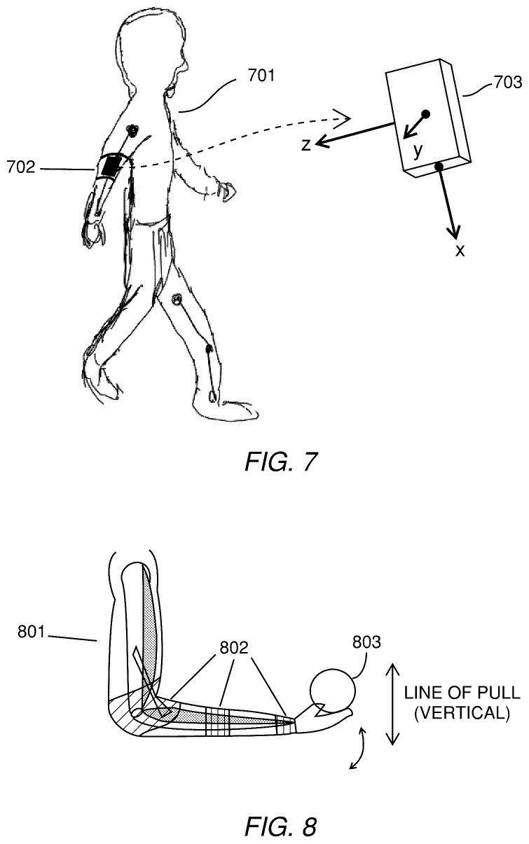

FIG. 7 depicts an active user 701 fitted with an exemplary embodiment comprising a wearable device 702. The wearable device 702 comprises at least one inertial measurement unit (IMU) 703 which includes one or more accelerometers which collect acceleration data in at least three degrees of freedom (in this case, X, Y, and Z). The IMU 703 is held to the user by, for example, an armband. The IMU may be, for example, a six-axis IMU with both accelerometer and gyroscope functionalities.

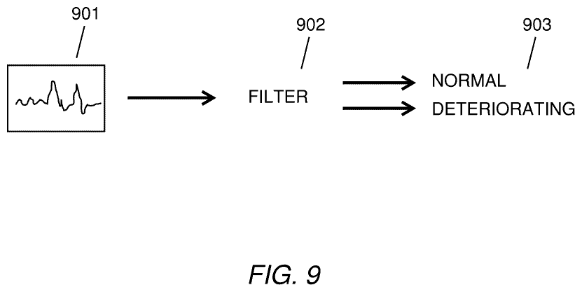

FIG. 8 depicts an arm 801 with three alternative placements 802 of a sleeve/armband of the wearable device 702. The placement of a wearable device such as device 702 may vary among users and context of use. In general, the optimal placement depends on the part or parts of the user for which it is desirable to monitor for muscle fatigue. As a non-limiting example, FIG. 8 depicts a weight 803 which the arm 801 is being used to lift. As many different muscles in the arm are generally employed in the act of lifting a weight 803 as depicted, any of the placements 802 may be sufficient. The signals of interest to be monitored for recognition of the onset of muscle fatigue may be sensed from any of multiple placements 802 along the arm 801. Other exercises or physical activity may warrant placement of sensors (e.g., IMU) on or proximal to other parts of the body. For instance, sensors may be placed on the legs, back, chest, or elsewhere.

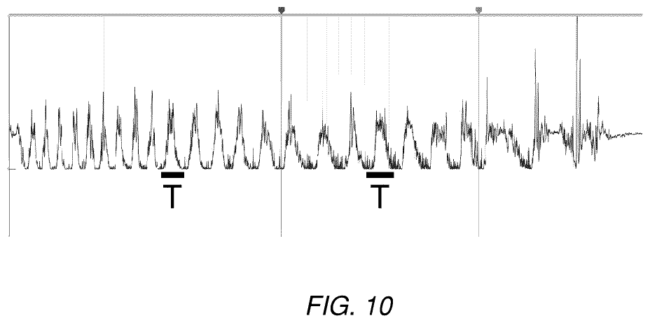

FIG. 9 is a flowchart of exemplary processing performed by the one or more processors 603 of FIG. 6. In general, the measurements 901 from the one or more sensors 601 may be subject to one or more forms of data processing 902 such as but not limited to filtering for frequencies above a predetermined threshold. Signals may be subject to low pass, high pass, and/or bandpass filters applied for observing the highest frequency of oscillation and spreading of oscillations (time delay), and the amplitude of oscillations within each period. The signals are continuously monitored over time for changes in the wave patterns. The one or more processors are configured to recognize one or more wave pattern changes in the measurements to make determinations 903 whether the most recent measurements are indicative of normal muscle function or deteriorating muscle function (i.e., the onset of muscle fatigue).

The following are exemplary wave pattern changes in monitored measurements which may be used to determine the onset of muscle fatigue:
(1) a period T increases from the previous average of periods (T_{A}) by greater than but not limited to 5%, wherein the period (T) is measured between peaks in at least two mutually orthogonal geometric planes,
(2) for an oscillation that has a peak frequency (F), the peak frequency (F) is greater than but not limited to 5% of the rolling average of peak frequencies of the previous two oscillations,
(3) a fundamental frequency of a given oscillation is greater than but not limited to a rate of 5% compared to the rolling average of fundamental frequencies of the previous ten oscillations, and
(4) a power frequency has a peak that is at least, but not limited to, two-fold over baseline.

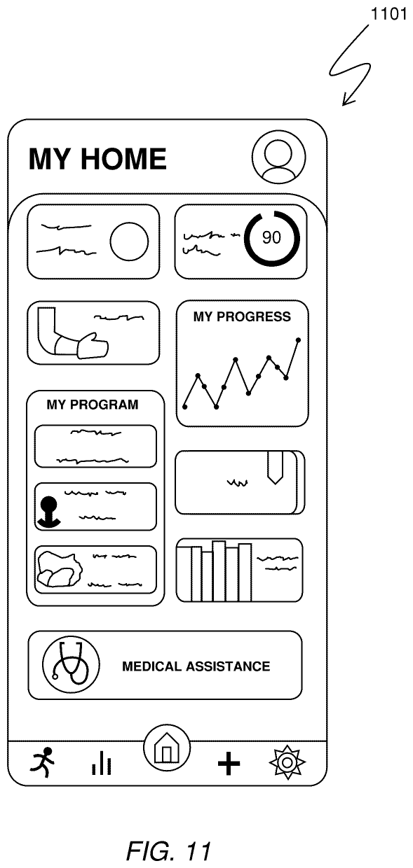

FIG. 10 illustrates the determination of a period (T) of the sample data originally presented in FIG. 1 as part of monitoring for a period increase over time.

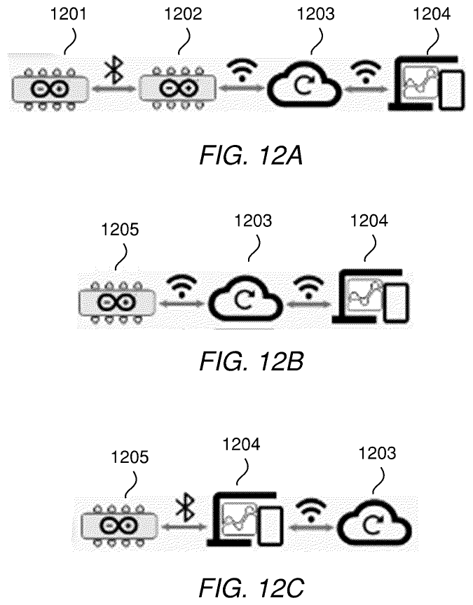

As already introduced in FIG. 6, an exemplary system 600 may include a feedback device 605 configured to produce one or more of a haptic, audial, and visual signal for conveying the alert to a user. FIG. 11 depicts the non-limiting example of an exemplary display 1101 of, e.g. a smartphone or tablet, which may be used to deliver a visual alert or other visual feedback to a user.

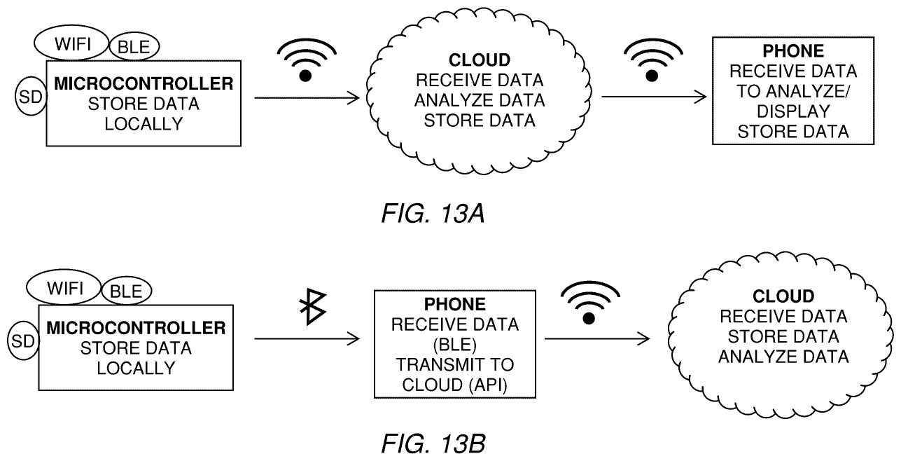

An exemplary system like system 600 may be configured as a collection of physically separate devices which coordinate to perform exemplary methods of this disclosure. Alternatively, some implementations of exemplary systems of this disclosure may be configured with some or all functions of exemplary methods performed by a single or just a few distinct devices. To this end, FIGS. 12A, 12B, and 12C present a few alternative system configurations.

FIG. 12A depicts a system configuration in which a peripheral device 1201 is connected by a wireless communication protocol such as Bluetooth to a central device 1202. the peripheral device 1201 may be, for example, a wearable like wearable device 702 or other object which is subject to transfer of forces by the user such as through contact. The central device 1202 may be, for example, a smartphone or smartwatch which may itself not be in close contact with the muscle(s) to be monitored. The central device 1202 has wireless communication capability such as a WiFi module which permit the central device to exchange data with IoT cloud services 1203. Data processing and determinations of exemplary methods of this disclosure may be performed by the central device, within remote server(s) of IoT cloud services, or a combination of these. Alerts may then be transferred back to the central device for display or other sensory output (haptic, visual, audial, etc.) via a mobile or web app 1204, for example.

FIG. 12B depicts an alternative configuration which consolidates hardware local to the user into a single device 1205. The single device 1205 may be configured to both perform movement data measurements (of acceleration and/or rotation) and communicate with IoT cloud services 1203. The device 1205 may be a special purpose wearable device, for example, or a more multiuse device such as smartphone worn on an arm or leg sleeve during activity monitoring, for example.

FIG. 12C depicts yet a further alternative configuration in which the mobile/web app is used to intermediate data transfer between the device 1205 and the IoT cloud services 1203.

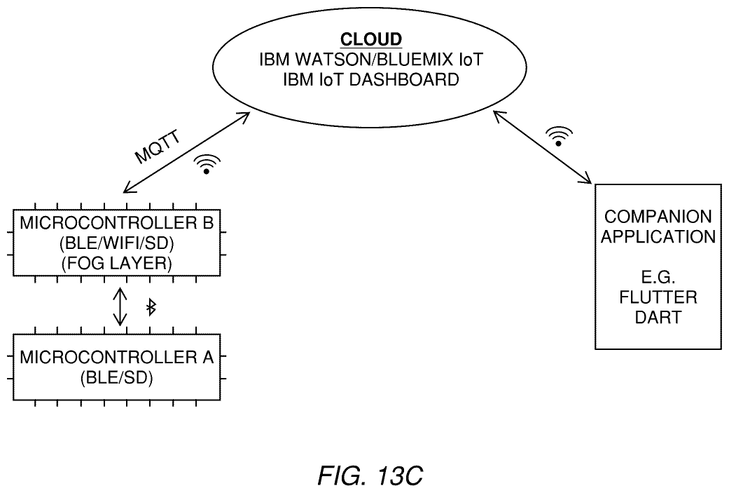

FIGS. 13A, 13B, and 13C show further exemplary system configurations with summarizes of functionalities of respective system components which coordinate with one other to perform exemplary methods of this disclosure.

As the figures illustrate, exemplary systems configured to monitor biomechanics of joints and muscles may include, for example, microcontroller(s) with IMU(s) and connectivity options such as Wi-Fi/BLE, an IoT cloud platform for data storage, and mobile/web application(s). Data analysis of IMU signals may be performed locally, remoted, or as combination of local and remote operations. That is, some embodiments may comprise processing and storing of data performed locally and displaying an application and/or sending data off to the IoT cloud services for storage and processing.

An exemplary device may comprise a single microcontroller or multiple microcontrollers or other types of microprocessors. An exemplary device may comprise at least two microcontrollers including a fog layer.

Elements of exemplary systems may communicate with one another via wired or wireless connections. Wireless connection protocols may include but are not limited to Wi-Fi and Bluetooth low energy (BLE). Components of exemplary systems may be integral with one another e.g. a sleeve with the microcontroller and receiver/transmitter hardware. In addition or in the alternative, components of exemplary systems may be separate and distinct from one another with means for communication and communication protocols between the components. Data may be streamed every 50 ms or 500 ms or 1000+ ms, for example. Uploaded data may include but is not limited to acceleration in one or more degrees of freedom e.g. acceleration in X and Y.

The present invention may be or include a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire. Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Where a range of values is provided in this disclosure, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are described.

As used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely”, “only”, and the like in connection with the recitation of claim elements, or use of a “negative” limitation.

As will be apparent to those of skill in the art upon reading this disclosure, each of the individual embodiments described and illustrated herein has discrete components and features which may be separated from or combined with the features of any of the other several embodiments without departing from the scope or spirit of the present invention. Any recited method can be carried out in the order of events recited or in any other order which is logically possible.

While exemplary embodiments of the present invention have been disclosed herein, one skilled in the art will recognize that various changes and modifications may be made without departing from the scope of the invention as defined by the following claims.

What is claimed is:

1. A method, comprising
monitoring frequencies in measurements of acceleration and/or rotation of at least one muscle;
detecting high frequency oscillations (HFOs) indicative of muscle fatigue preceding muscle failure in the monitored frequencies; and
in response to the detection of the HFOs, sending an alert before occurrence of muscle failure.

2. The method of claim 1, wherein the frequencies which are monitored include frequencies greater than 80 Hz.

3. The method of claim 1, wherein the measurements are in multiple degrees of freedom.

4. The method of claim 1, further comprising collecting the measurements with at least one accelerometer and/or at least one gyroscope.

5. The method of claim 1, further comprising collecting the measurements with at least one inertial measurement unit (IMU).

6. The method of claim 1, wherein the detecting comprises recognizing at least one of the following wave pattern changes in the measurements:
a period T increases from the previous average of periods (T_{A}) by greater than but not limited to 5%, wherein the period (T) is measured between peaks in at least two mutually orthogonal geometric planes,
for an oscillation that has a peak frequency (F), the peak frequency (F) is greater than but not limited to 5% of the rolling average of peak frequencies of the previous two oscillations,
a fundamental frequency of a given oscillation is greater than but not limited to a rate of 5% compared to the rolling average of fundamental frequencies of the previous ten oscillations, and
a power frequency has a peak that is at least, but not limited to, two-fold over baseline.

7. The method of claim 1, wherein the detecting comprises recognizing at least one wave pattern change in the acceleration measurements, wherein the at least one wave pattern change comprises: a period T increases from the previous average of periods (T_{A}) by greater than but not limited to 5%, wherein the period (T) is measured between peaks in at least two mutually orthogonal geometric planes.

8. The method of claim 1, wherein the detecting comprises recognizing at least one wave pattern change in the acceleration measurements, wherein the at least one wave pattern change comprises: for an oscillation that has a peak frequency (F), the peak frequency (F) is greater than but not limited to 5% of the rolling average of peak frequencies of the previous two oscillations.

9. The method of claim 1, wherein the detecting comprises recognizing at least one wave pattern change in the acceleration measurements, wherein the at least one wave pattern change comprises: a fundamental frequency of a given oscillation is greater than but not limited to a rate of 5% compared to the rolling average of fundamental frequencies of the previous ten oscillations.

10. The method of claim 1, wherein the detecting comprises recognizing at least one wave pattern change in the acceleration measurements, wherein the at least one wave pattern change comprises: a power frequency has a peak that is at least, but not limited to, two-fold over baseline.

11. A system, comprising
one or more sensors configured to measure acceleration and/or rotation of at least one muscle; and
one or more processors configured to
monitor frequencies in the measurements of acceleration and/or rotation of the at least one muscle;
detect high frequency oscillations (HFOs) indicative of muscle fatigue preceding muscle failure in the monitored frequencies; and
in response to the detection of the HFOs, send an alert before occurrence of muscle failure.

12. The system of claim 11, wherein the one or more sensors are configured together in at least one inertial measurement unit (IMU).

13. The system of claim 11, wherein the one or more sensors includes at least one accelerometer configured to measure acceleration.

14. The system of claim 11, wherein the one or more sensors includes at least one gyroscope configured to measure rotation.

15. The system of claim 11, further comprising a feedback device configured to produce one or more of a haptic, audial, and visual signal for conveying the alert to a user.

16. The system of claim 11, wherein a sampling rate of the one or more sensors is at least 9600 Hz.

17. The system of claim 11, wherein the frequencies which are monitored include frequencies greater than 80 Hz.

18. The system of claim 11, wherein the measurements are in multiple degrees of freedom.

19. The system of claim 11, wherein the detecting comprises recognizing at least one of the following wave pattern changes in the measurements:
a period T increases from the previous average of periods (T_{A}) by greater than but not limited to 5%, wherein the period (T) is measured between peaks in at least two mutually orthogonal geometric planes,
for an oscillation that has a peak frequency (F), the peak frequency (F) is greater than but not limited to 5% of the rolling average of peak frequencies of the previous two oscillations,
a fundamental frequency of a given oscillation is greater than but not limited to a rate of 5% compared to the rolling average of fundamental frequencies of the previous ten oscillations, and
a power frequency has a peak that is at least, but not limited to, two-fold over baseline.

20. The system of claim 11, wherein the detecting comprises recognizing at least two of the following wave pattern changes in the measurements:
a period T increases from the previous average of periods (T_{A}) by greater than but not limited to 5%, wherein the period (T) is measured between peaks in at least two mutually orthogonal geometric planes,
for an oscillation that has a peak frequency (F), the peak frequency (F) is greater than but not limited to 5% of the rolling average of peak frequencies of the previous two oscillations,
a fundamental frequency of a given oscillation is greater than but not limited to a rate of 5% compared to the rolling average of fundamental frequencies of the previous ten oscillations, and
a power frequency has a peak that is at least, but not limited to, two-fold over baseline.
